(19) United States
(12) Patent Application Publication
(10) Pub. No.: US 2021/0302418 A1
(43) Pub. Date: Sep. 30, 2021

(54) ULTRASENSITIVE DETECTION OF VIRUS PARTICLES AND VIRUS-LIKE PARTICLES

(71) Applicant: FORSCHUNGSZENTRUM JUELICH GMBH, Juelich (DE)

(72) Inventor: Dieter WILLBOLD, Juelich (DE)

(21) Appl. No.: 16/348,194

(22) PCT Filed: Nov. 9, 2017
(86) PCT No.: PCT/EP2017/078777

§ 371 Date: May 8, 2019

(30) Foreign Application Priority Data
Nov. 9, 2016 (DE) 102016121455.8

(51) Int. Cl. G01N 33/543; G01N 33/569; G01N 33/58

(52) U.S. Cl. CPC G01N 33/54393; G01N 33/582; G01N 33/56983

(57) ABSTRACT

The present invention relates to a method for quantitatively and/or qualitatively determining virus particles containing at least one binding site for a capture molecule and at least one binding site for a probe, to a kit for carrying out said method, and to various uses.

The present invention relates to a method for quantitatively and/or qualitatively determining virus particles containing at least one binding site for a capture molecule and at least one binding site for a probe, to a kit for carrying out said method, and to various uses.

Viruses are defined as infectious particles which can only propagate within suitable host cells. Viruses are constructed from nucleic acid (DNA or RNA), proteins and, in some cases, lipids as well. They also include bacteriophages, which infect bacteria. Since they all do not have independent replication or their own metabolism, they cannot be classed as living organisms according to prevailing opinion. Individual virus particles consist of a genome of the abovementioned nucleic acid and of a protein coat. Some have an additional coat. Such a complete, infectious virus particle represents the extracellular form of the virus and is also referred to as a virion. Owing to the small size of the infectious virus particles, between 15 nm and 300 nm depending on the species, only a few direct detection methods are known to date; the majority of detections are based on the symptoms of the infected cells or infected living organisms. When pathogens, such as virus particles for example, actively or passively penetrate, remain and subsequently propagate in an organism, this is generally referred to as an infection. If the host cells can be classed as prokaryotes, the infectious virus particles are called bacteriophages.

Owing to the small size of the particles, electron microscopy is used as the hitherto only direct detection of virus particles. This involves detection of viruses on the basis of their properties as particles. In addition to the high costs for the appropriate equipment, electron microscopy only makes it possible to distinguish between different virus families, but does not make it possible to determine corresponding subspecies. Furthermore, the samples must be chemically or physically fixed, with the result that functional proteins are no longer available or epitopes are denatured or masked. In addition, the presence of a high number of virus particles is necessary.

According to the state of the art, viruses or viral components are detected by means of functional, antibody-based or genome-based techniques, such as, for example, PCR-based multiplication of viral RNA or DNA and identification by means of hybridization-dependent probes.

Functional viruses are detected by means of the plaque assay with utilization of the cytopathic effect. Inactivated viruses or noninfectious particles are not registered. Genome-based techniques quantify more the amount of viral RNA/DNA than the amount of virus particles.

Continuous epitopes of viral proteins can be detected with antibodies in Western blotting.

In the case of strong suspicion of a viral infection and when other detection methods fail to provide a positive result, the PCR technique is used. This involves amplifying the genome by means of the polymerase chain reaction with use of virus-specific primers. In the case of RNA viruses, this requires the transcription of RNA into DNA by means of reverse transcriptase. The amplicon is detected by hybridization by specific probes or by nonspecific staining of the amplified DNA and size-dependent identification following gel electrophoresis.

PCR-based methods must be laboriously calibrated, are not strictly quantitative and highly prone to contamination, which may lead to a false-positive result. In reality, they determine the presence of viral DNA/RNA or sometimes even only parts thereof. In addition, sample components which inhibit the PCR reaction may cause a false-negative result. Therefore, the samples must always be purified before they can be analyzed.

Immunoassays, which detect endogenous antiviral antibodies, can generally lead to a positive result only weeks after infection (diagnostic gap). ELISA methods generally detect only the presence of subfragments of a virus and not intact particles.

It is an object of the present invention to provide an ultrasensitive detection for virus particles. It is a further object to provide a method which allows the detection of virus particles and virus-like particles in any sample and in a smallest possible number, thus even individual detection in any sample, especially for nonculturable viruses as well. As a result, it is possible to carry out a detection of viral infections and also contamination in any sample.

Furthermore, it is intended that not only a qualitative detection of virus particles be possible, but also a quantification and characterization of virus particles in any sample. It is thereby intended that, firstly, a direct and absolute quantification of the particle number and, secondly, an accurate characterization of the virus particles be ensured and that typing thus be made possible. It is intended that the results be usable in therapy-accompanying diagnostics, differential diagnostics and/or analysis of virus assembly.

Furthermore, it is intended that the method also ensure the detection of protein-protein interactions, i.e., of host-virus interactions.

It is intended that the detection be possible with a few simple steps directly from any sample such as, for example, ex vivo from body fluids or autopsy or biopsy material, organs, but also samples from the environment, such as, for example, water samples, plant samples and soil samples, and also foods.

These objects are achieved by a method for quantitatively and/or qualitatively determining virus particles containing at least one binding site for a capture molecule and at least one binding site for a probe. The method comprises the following steps:
a) immobilizing capture molecules on a substrate,
b) contacting the virus particles with the capture molecules,
c) immobilizing the virus particles on the substrate by binding to capture molecules,
d) contacting the virus particles with the probes and
e) binding the probes to the virus particles,
   wherein the probes are capable of emitting a specific signal and steps b) and d) can be carried out simultaneously or d) before b).

If steps b) and d) are carried out simultaneously, steps c) and e) are thus also carried out simultaneously.

In the further variant in which step d) is carried out before step b), virus particles labeled with probes are thus immobilized on the substrate in step c). Consequently, step e) is thus also carried out before steps b) and c).

In the context of the present invention, “quantitative determination” means first of all the determination of the concentration of the virus particles, thus also the determination of their presence or absence.

Preferably, quantitative determination also means the selective quantification of certain virus types. Such a quantification can be controlled via the appropriate probes.

In the context of the present invention, “qualitative determination” means the characterization of the virus particles, such as, for example, the determination of the form.

The virus particles are labeled with one or more probes serving for detection. In one variant, at least two, three, four, five, six, seven or more probes are used. In a further variant, two, three, four, five, six, seven or more different probes are used.

The probes contain a molecule or molecule part which has an affinity for virus particles and which recognizes a binding site of the virus particle and binds thereto. In addition, the probes contain at least one detection molecule or molecule part which is covalently bonded to the molecule or molecule part having an affinity for virus particles and is detectable and measurable by means of chemical or physical methods.

In one alternative, the probes can comprise identical affinity molecules or molecule parts with different detection molecules (or parts). In a further alternative, different affinity molecules or molecule parts can be combined with different detection molecules or parts, or alternatively different affinity molecules or parts can be combined with identical detection molecules or parts. It is also possible to use mixtures of various probes.

The use of multiple different probes coupled to different detection molecules or molecule parts increases, firstly, the specificity of the signal (correlation signal); secondly, this allows the identification of virus particles differing in one or more features. This allows a selective quantification and characterization of the virus particles.

In one embodiment, a spatially resolved determination of the probe signal is carried out, i.e., a spatially resolved detection of the signal emitted by the probe. Accordingly, this embodiment of the invention excludes methods based on a non-spatially resolved signal, such as ELISA or sandwich ELISA. This also includes ELISA-like methods, i.e., all methods which are based on a non-spatially resolved signal and which are, however, based on “bulk” measurements, in other words: ensemble measurements; thus all immunoassays, irrespective of whether the detection is based on an enzymatic color reaction, or on detection of fluorescence or of magnetically or radioactively labeled probes or antibodies, if what is detected is not the signal of individual particles, but of entire volume segments.

The method according to the invention is further characterized by the following features:

The determination of intact, undestroyed virus particles (i.e., undestroyed viruses); the determination of the number of said particles and/or form; investigation or analysis of individual particles, thus no ensemble measurement; determination and analysis of low concentrations of 100 particles/μl or less; differentiation between empty virus coats and virus particles containing, besides the coat, further constituents such as, for example, DNA, RNA, proteins different to those of the coat;

In the detection, a high spatial resolution is advantageously not essential, however. In one embodiment of the method according to the invention, sufficient data points are collected to allow the detection of a virus or virus-like particle against a background signal caused, for example, by instrument-specific noise, other nonspecific signals or nonspecifically bound probes. In this way, as many values as spatially resolved events, such as pixels for example, are present are read out (readout values). Owing to the spatial resolution, each event is determined against the respective background and is thus an advantage over ELISA methods with no spatially resolved signal.

In one embodiment, the spatially resolved determination of the probe signal is based on the investigation of a small volume element in comparison with the volume of the sample, within the range from a few femtoliters to below one femtoliter, or of a volume region above the contact surface of the capture molecules at a height of 500 nm, preferably 300 nm, particularly preferably 250 nm and in particular 200 nm.

In the context of the invention, virus particles are selected from the group containing or consisting of virus, virion, bacteriophage and parts or fragments thereof. Virus-like particles are, for example, virus coats, parts or fragments thereof which are incapable of replication. In the context of the invention, the term virus also encompasses virus-like particles and also, in each case, parts or fragments of viruses and/or virus-like particles.

Taxonomically, viruses can be divided into viruses which have a capsid, i.e., a coat of proteins, and viruses which have a coat of lipids, a lipid bilayer membrane containing embedded viral proteins. In the context of the invention, all virus particles according to the invention can be divided to that effect, thus into particles which have a coat or parts of a coat containing lipids and possibly additionally a coat or parts of a coat composed of proteins, and particles which merely have a coat or parts of a coat composed of proteins.

Owing to the possibility of detecting and analyzing individual particles, it is possible with appropriate selection of different catchers and probes to also analyze different viruses in parallel in one sample. Thus, the method can also be used for differential diagnosis.

The method is not carried out in and/or on the human body, but ex vivo, thus in vitro.

According to the invention, the parts or fragments of the virus particles are parts containing at least two binding sites.

In one embodiment, the material of the substrate is selected from the group containing or consisting of plastic, silicon and silicon dioxide. In a preferred alternative, the substrate used is glass.

In a further embodiment of the invention, the capture molecules are covalently bonded to the substrate.

To this end, what is used in one alternative is a substrate having a hydrophilic surface. In one alternative, this is achieved by the application of a hydrophilic layer to the substrate prior to step a). Thus, the capture molecules bind covalently to the substrate or to the hydrophilic layer with which the substrate is loaded.

The hydrophilic layer is a biomolecule-repellent layer, meaning that the nonspecific binding of biomolecules to the substrate is minimized. The capture molecules are immobilized on said layer, preferably covalently. Said capture molecules have an affinity with respect to a feature of the virus particles. The capture molecules can all be identical, or be mixtures of different capture molecules. In one alternative, the capture molecules and probes that are used are the same molecules; preferably, the capture molecules do not contain detection molecule or molecule parts.

In one embodiment, the hydrophilic layer is selected from the group containing or consisting of PEG, poly-lysine, preferably poly-D-lysine, and dextran or derivatives thereof, preferably carboxymethyl-dextran (CMD). Derivatives in the context of the invention are compounds which differ from the parent compounds in some substituents, the substituents being inert with respect to the method according to the invention.

In one embodiment, the surface of the substrate is first hydroxylated and then functionalized with suitable chemical groups, preferably amino groups, prior to application of the hydrophilic layer. In one alternative, this functionalization with amino groups is achieved by contacting the substrate with aminosilanes, preferably APTES (3-aminopropyltriethoxysilane), or with ethanolamine.

To prepare the substrate for the coating, one or more of the following steps are carried out:
washing of a substrate composed of glass or of a glass slide in an ultrasonic bath or plasma cleaner, or alternatively incubate in 5 M NaOH for at least 3 hours,
rinsing with water and subsequent drying under nitrogen or under vacuum,
immersion in a solution composed of concentrated sulfuric acid and hydrogen peroxide in the ratio of 3:1 to activate the hydroxyl groups,
rinsing with water up to a neutral pH, then with ethanol and drying under a nitrogen atmosphere,
immersion in a solution containing 3-aminopropyltriethoxysilane (APTES) (1-7%), preferably in dry toluene, or a solution of ethanolamine,
rinsing with acetone or DMSO and water and drying under a nitrogen atmosphere.

In one alternative, the substrate is contacted with aminosilanes, preferably APTES, in the gas phase; the optionally pretreated substrate is thus subjected to vapor deposition with the aminosilanes.

For the coating with dextran, preferably carboxymethyl-dextran (CMD), the substrate is incubated with an aqueous solution of CMD (in a concentration of 10 mg/ml or 20 mg/ml) and optionally N-ethyl-N-(3-dimethylaminopropyl)carbodiimide (EDC) (200 mM) and N-hydroxysuccinimide (NHS) (50 mM) and then washed.

In one variant, the carboxymethyl-dextran is covalently bonded to the glass surface which was first hydroxylated and then functionalized with amine groups, as described above.

The substrate used can also be microtiter plates, preferably with a glass base. Since the use of concentrated sulfuric acid is not possible when using polystyrene frames, the glass surface is, in one embodiment of the invention, activated analogously to Janissen et al. (Colloids Surf B Biointerfaces, 2009, 71(2), 200-207).

What are immobilized on this hydrophilic layer (preferably covalently) are capture molecules which have an affinity with respect to a feature (e.g., proteins) of the virus-like or virus particles to be detected. The capture molecules can all be identical or mixtures of various capture molecules.

In one embodiment of the present invention, the capture molecules (preferably antibodies) are immobilized on the substrate optionally after an activation of the CMD-coated support by a mixture of EDC/NHS (200 and 50 mM, respectively).

Remaining carboxylate end groups, to which no capture molecules have been bonded, can be deactivated. To deactivate said carboxylate end groups on the CMD spacer, ethanolamine in DMSO is used. Prior to the application of the samples, the substrates or supports are optionally rinsed with PBS.

The sample to be measured is contacted with the thus prepared substrate and, if necessary, incubated. The sample to be investigated that is used can be endogenous liquids or tissue. In one embodiment of the present invention, the sample is selected from cerebrospinal fluid (CSF), blood, plasma and urine. Foods and swabs of objects are used as samples too, however. The samples can pass through different processing steps known to a person skilled in the art.

In one embodiment of the present invention, the sample is directly applied on the substrate (uncoated substrate), optionally by covalent bonding on the optionally activated surface of the substrate.

In one variant of the present invention, the sample is pretreated by one or more of the following methods:
heating of the sample,
one or more freeze-thaw cycles,
mechanical disruption,
homogenization of the sample,
dilution with water or buffer,
treatment with enzymes, for example proteases,
nuclease, lipases,
centrifugation,
precipitation,
competition with probes in order to displace any antibodies present.

Preferably, the sample is contacted with the substrate directly and/or without pretreatment.

Nonspecifically bound substances can be removed by wash steps.

In a further step, immobilized virus-like particles or virus particles are labeled with one or more probes serving for further detection. As described above, the individual steps can also be carried out according to the invention in a different order.

By means of suitable wash steps, excess probes not bound to the virus particles are removed.

In one alternative, said excess probes are not removed. As a result, the last wash steps are omitted and there is also no shift in equilibrium in the direction of the dissociation of the virus particle-probe complexes or bonds. Owing to the spatially resolved detection, the excess probes are not registered in the evaluation and do not impair the measurement.

In one variant, the virus particle-capture molecule complexes are chemically fixed in addition to the immobilization on the substrate, i.e., virus particle and capture molecule are connected to one another by chemical bonds, preferably covalent bonds, in addition to the linkage via the binding site, meaning that dissociation is prevented.

In one alternative, probe-virus particle-capture molecule complexes are chemically fixed in addition to the immobilization on the substrate, i.e., probe(s), virus particle and capture molecule are connected to one another by chemical bonds, preferably covalent bonds, in addition to the linkage via the binding site, meaning that dissociation is prevented.

In another alternative, probe-virus particle complexes are chemically fixed, i.e., virus particle and probe(s) are connected to one another by chemical bonds, preferably covalent bonds, in addition to the linkage via the binding site, meaning that dissociation is prevented. This is followed by immobilization on the substrate.

In one embodiment, the binding sites of the virus particles are epitopes and the capture molecules and/or probes are antibodies or aptamers or combinations thereof. In one variant, capture molecules and/or probes are antibodies. In one variant of the present invention, capture molecules and probes can be identical.

In one embodiment of the present invention, capture molecules and probes differ. For example, different antibodies can be used as capture molecules and probes. In a further embodiment of the present invention, capture molecules and probes are used which are identical to one another with the exception of any (dye) label. In one alternative of the present invention, various probes are used which are identical to one another with the exception of any (dye) label. In further alternatives of the present invention, at least two or more different capture molecules and/or probes are used which contain different antibodies and optionally also have different dye label.

For the detection, the probes are characterized such that they emit an optically detectable signal selected from the group consisting of fluorescence emission, bioluminescence emission and chemiluminescence emission and also absorption.

In one alternative, the probes are thus labeled with fluorescent dyes. The fluorescent dyes used can be the dyes known to a person skilled in the art. Alternatively, it is also possible to use fluorescent biomolecules, preferably GFP (green fluorescence protein), conjugates and/or fusion proteins thereof, and also quantum dots.

For the later quality control of the surface, for example evenness of the coating with capture molecules, it is possible to use capture molecules labeled with fluorescent dyes. To this end, preference is given to using a dye which does not interfere with the detection. What is possible as a result is a subsequent structure check and also a normalization of the measurement results.

The immobilized and labeled virus-like or virus particles are detected by means of imaging of the surface (e.g., laser microscopy). A highest possible spatial resolution ascertains a high number of pixels, the result being that the sensitivity and the selectivity of the assay can be increased, since structural features can be concomitantly imaged and analyzed. Thus, the specific signal increases against the background signal (e.g., nonspecifically bound probes).

The detection is preferably carried out using confocal fluorescence microscopy, fluorescence correlation spectroscopy (FCS), especially in combination with cross correlation and laser scanning microscope (LSM). In one alternative of the present invention, the detection is carried out using a confocal laser scanning microscope.

In one embodiment of the present invention, a laser focus, as used for example in laser scanning microscopy (LSM), or an FCS (fluorescence correlation spectroscopy system) is used to this end, as are the corresponding super-resolution variants such as, for example, STED, PALM or SIM.

In a further embodiment, the detection can be achieved by means of spatial-resolution fluorescence microscopy, preferably by means of a TIRF microscope, and also the corresponding super-resolution variants thereof, such as, for example, STORM, dSTORM.

Thus, preferably LSM and/or TIRF, particularly preferably TIRF, is used for the detection.

In contrast to ELISA, these methods give rise to as many readout values as spatially resolved events (e.g., pixels) are present. Depending on the number of different probes, this information is even multiplied. This multiplication applies to any detection event and leads to an information gain, since it discloses further properties (e.g., second feature) about virus-like or virus particles. Owing to such a setup, the specificity of the signal can be increased for any event.

The probes can be selected such that the presence of individual virus constituents (e.g., individual coat/capsid protein molecules) do not influence the measurement result. The probes can be selected such that virus species and subspecies (serotypes) can be determined for any individual virus particle. Additional probes can be selected such that it is possible to distinguish between DNA/RNA-containing and “empty” virus coats, for example by means of DNA/RNA-binding fluorophores (EtBr, EtI).

For the evaluation, the spatially resolved information (e.g., fluorescence intensity) of all the probes used and detected is used for determining, for example, the number of virus-like or virus particles, the size thereof and the features thereof. At the same time, it is possible, for example, for also algorithms of background minimization and/or also intensity thresholds to be used for further evaluation and also pattern recognition. Further image analysis options include, for example, the search for local intensity maxima in order to obtain the number of detected virus particles from the image information.

To make the assay results comparable with one another (across removals, times and experimenters), standards (internal and/or external) can be used.

In one embodiment of the invention, what are thus used are standards, especially standards as described in WO 2016/146093 A. These are preferably used as calibration standards and/or internal standards. According to the invention, what are used to this end are standards which correspond to the size of the viruses, thus between 10 and 500 nm in diameter, preferably 20-200 nm diameter. Preference is give to using, as base body, an inorganic nanoparticle to which parts of the virus-particle coat proteins containing or consisting of epitopes are bound in a method according to the abovementioned WO 2016/146093 A.

In one embodiment, such a standard is prepared in the following steps:

A) providing an inorganic nanoparticle having the size of the virus particle to be analyzed (10-500 nm, preferably 20-200 nm),
B) forming free amino groups or free carboxy groups on the surface of the nanoparticle in order to functionalize the nanoparticle surface to form an amine- or carboxy-functionalized nanoparticle,

### C)

i) binding of maleimido-spacer-carboxylic acid to the free amino groups in step B),
or
ii) converting the free carboxy groups in step B) into NHS esters,
D) binding coats or parts of coats containing or consisting of at least one epitope
i) to the maleimido-spacer-carboxylic acids via a sulfhydryl group at the free end of the coats or parts of coats containing or consisting of at least one epitope,
or
ii) to the NHS esters via the amino group at the free end of the coats or parts of coats containing or consisting of at least one epitope.

Method as described above, characterized by Stober synthesis in step A) to prepare a silica nanoparticle.

Method as described above, characterized by silanization of the surface with aminopropyltriethoxysilane in ethanol to form free amino groups in step B).

Method according to any of the above-described methods, characterized by reaction of the amino groups with succinic anhydride to form free carboxy groups in step B).

Method according to any of the above-described methods, in which the maleimido-spacer-carboxylic acid is converted into an NHS ester before it is bound in step C) i) to the free amino group of step B).

Method according to any of the above-described methods, characterized by a reaction of the maleimido-spacer-carboxylic acid from step C) i) or of the free carboxy groups from step B) with 1-ethyl-3-(3-dimethylaminopropyl)carbodiimide and/or N-hydroxysuccinimide for conversion into an NHS ester before step C) i) or in step C) ii).

Method according to any of the above-described methods, characterized by covalent bonding of the amino group of the coats or parts of coats containing or consisting of at least one epitope with the NHS ester in step D ii).

Method according to any of the above-described methods, characterized in that a streptavidin molecule is arranged on the NHS ester of the nanoparticle after the performance of step C) ii) and a biotin group is arranged on the shells or parts of shells containing or consisting of at least one epitope of the protein aggregate before step D).

In one embodiment of the present invention, what can be achieved using the method according to the invention is the number of virus particles in a suspension without use of calibration standards or external or internal standards. To this end, what is optionally first prepared is a concentration series of serially diluted virus particles. Said series is analyzed using the method according to the invention. In this connection, what are consecutively recorded per reaction chamber at different positions (e.g., at 5×5 positions) are in each case two images in two fluorescence channels (excitation/emission=635/705 nm and 488/525 nm). For both channels, what can be selected is the maximum laser output (100%), an exposure time of 500 ms and a gain value of 800. The image data are then evaluated. For this purpose, the intensity thresholds for each channel are ascertained on the basis of the negative control. For said threshold, all images of the negative control are averaged for each channel and what is ascertained is that intensity value above which only 0.1% of the total pixels (ergo 1000 pixels) are present. In the evaluation step, the intensity threshold is first applied for each image in each channel and images of the same position are then compared with one another in both values. What are counted per image are only those pixels in which, in both channels, the pixels at the exact same position are above the intensity threshold of the channel. Lastly, the number of pixels is averaged over all images in each reaction chamber and, afterwards, the mean values of the average pixel numbers of the replicate values are ascertained and the standard deviation is specified.

In a further evaluation, what are multiplied for each recording are the grayscale values of each corresponding pixel from both images in which fluorescence color channels were obtained. The resultant image, or the grayscale value matrix, is smoothed on the basis of a 2D Gaussian function (“imgaussfilt”) with a standard deviation of a few pixels, for example 4 pixels. This is followed by the determination of the local maxima on the basis of the function “imregionalmax”. The number of local maxima above a threshold corresponds here to the number of particles, the threshold corresponding to the threshold ascertained above. In this way, the number of particles is determined for each recording and the mean value is formed over reaction chambers of the same concentration. This means that it is possible to specify the absolute number of virus particles. A precise description can be gathered from Examples 5 and 6.

The present invention also provides methods for determining the absolute number of virus particles in a suspension without use of standard, especially without use of calibration standards or external or internal standards.

The present invention also provides a kit containing one or more of the following components:

substrate, optionally with hydrophilic surface, capture molecule, probe, substrate with capture molecule, solutions, buffers.

The compounds and/or components of the kit of the present invention can be packaged in containers, optionally with/in buffers and/or solution. Alternatively, some components can be packaged in the same container. In addition to this or as an alternative to this, one or more of the components could be adsorbed to a solid support, such as, for example, a glass plate, a chip or a nylon membrane, or to the well of a microtiter plate. The kit can further contain instructions for use of the kit for any of the embodiments.

In a further variant of the kit, the above-described capture molecules are immobilized on the substrate. In addition, the kit can contain solutions and/or buffers. To protect the biomolecule-repellent surface (e.g., dextran surface) and/or the capture molecules immobilized thereon, they can be overlaid with a solution or a buffer. In one alternative, the solution contains one or more biocides, which increase the shelf life of the surface.

The present invention further provides for the use of the method according to the invention for the detection of virus particles and virus-like particles in all samples, for the quantification (titer determination) of virus particles and virus-like particles, detection of a viral infection, detection of a viral contamination, use in the development of active antiviral ingredients, direct and absolute quantification of particle number, therapy-accompanying diagnostics (target engagement), analysis of virus assembly, differential diagnostics, detection of protein-protein interaction (host/virus) and/or virus typing.

The present invention further provides for the use of the method according to the invention for the monitoring of therapies of infectious diseases and for the monitoring and/or checking of the efficacy of active ingredients and/or treatment methods, for example via the determination of the titer of virus particles or virus-like particles. This can be used in clinical tests and trials and in therapy monitoring. To this end, samples are measured in accordance with the method according to the invention and the results compared.

The present invention further provides for the use of the method according to the invention for the determination of the efficacy of active ingredients against viruses, in which method the results of samples are compared with one another. The samples are body fluids, collected before and/or after administration of the active ingredients or performance of the treatment method, or at different time points thereafter. According to the invention, the results are compared with a control which was not subjected to the active ingredient and/or treatment method. Active ingredients and/or the dose thereof and/or treatment methods are selected on the basis of the results.

The present invention further provides for the use of the method according to the invention for determining whether a person is included in a clinical trial. To this end, samples are measured in accordance with the method according to the invention and the decision is made with reference to a limit value.

### EXAMPLES

### Example 1

The experiment was carried out in commercially available 3D NHS microtiter plates (PolyAn GmbH) containing 384 reaction chambers (RCs). The RCs of the microtiter plates were coated with antibodies: clone anti-gp8-E1 (prod. #ABIN793840, lot #77410, antibodies-online.com) as capture molecule (15 μl; μg/ml in 100 mM MES, pH=4.7; incubation overnight). Thereafter, the RC was subjected to a wash program consisting of washing and aspiration three times, in each case with phosphate-buffered saline (PBS) containing 0.1% Tween 20 and PBS. In the next step, the RCs were coated with 50 μl of Smartblock (Candor Bioscience GmbH) at room temperature (RT) for 1 h and were subjected to the above-described wash program again after this time had passed. Thereafter, 15 μl of the sample, in sequential dilution in human EDTA blood plasma in quadruplicate, were in each case loaded in RCs and incubated at RT. After incubation overnight, the RCs were washed using the wash program, the RCs were sucked dry and detection antibodies were loaded. The detection antibodies were in each case labeled with one type of fluorescent dye. The antibody RL-ph1 (prod. #LS-C146750, LifeSpan BioScience) was labeled with the fluorescent dye CF488 and the antibody LRL-ph2 (prod. #LS-C146751, lot #76955, LifeSpan BioScience) was labeled with the fluorescent dye CF633. The detection antibodies were diluted together in PBS to give a final concentration of 1.25 ng/ml for each antibody. 15 μl of antibody solution were loaded per RC and incubated at room temperature for 1 h. After this time had passed, the plate was washed 5 times with PBS containing 0.1% Tween 20 and 5 times with PBS. After complete suction, the RCs were filled with 20 μl of water and the plate was sealed with a film.

The measurement was carried out in a TIRF microscope (Leica) with a 100× oil immersion objective. For this purpose, the glass base of the microtiter plate was generously coated with immersion oil and the plate was introduced into an automated stage of the microscope. Thereafter, what was consecutively recorded per RC at 5×5 positions was in each case two images in two fluorescence channels (excitation/emission=635/705 nm and 488/525 nm). For both channels, what was selected was the maximum laser output (100%), an exposure time of 500 ms and a gain value of 1300. The image data were then evaluated.

For the analysis, a quadratic ROI of 800 was first used. This means that the outermost 100 pixels on each side of each image were in each case not included in the evaluation, meaning that a 1000×1000 pixels image gives rise to an 800×800 pixels image. In the next step, intensity thresholds for each channel were ascertained on the basis of the negative control. For said threshold, all images of the negative control were averaged for each channel and what was ascertained was that intensity value above which only 0.1% of the total pixels (ergo 640 pixels) are present. In the evaluation step, the intensity threshold was first applied for each image in each channel and images of the same position were then compared with one another in both values. What were counted per image were only those pixels in which, in both channels, the pixel at the exact same position is above the intensity threshold of the channel. Lastly, the number of pixels was averaged over all images in each RC and, afterwards, the mean values of the average pixel numbers of the replicate values were ascertained and the standard deviation was specified.

The results are summarized in FIG. 1.

FIG. 1 shows a concentration series of serially diluted M13 phages (Ph.D.-7 Phage Display Library; prod. #E8102L, lot #0061005, New England BioLabs) in negative human EDTA blood plasma. The figure shows a linear relationship between the measurement signal and the concentration of M13 phages over 5 log dilution steps and a distinguishability of the lowest concentration in relation to the background.

### Example 2

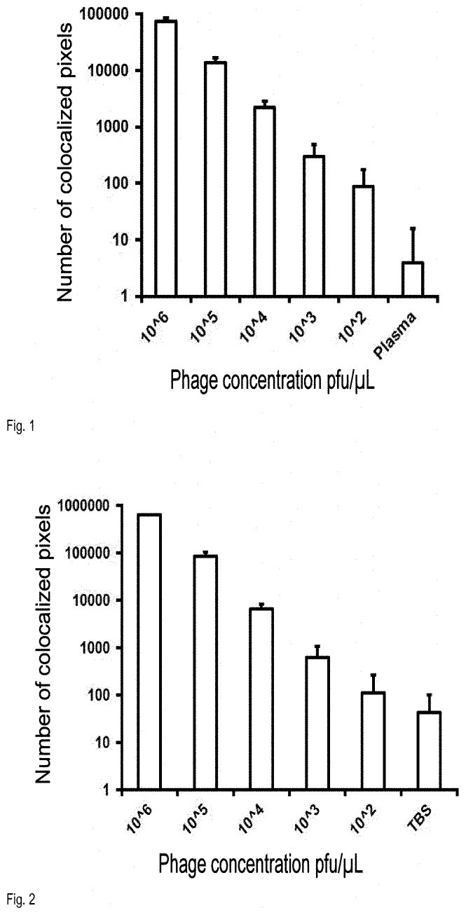

The experiment, the result of which is summarized in FIG. 2, was carried out analogously to Example 1, with the difference that the phages were diluted in TRIS-buffered saline instead of in human blood plasma. This also gave rise to different intensity thresholds, which however were ascertained using the same rule (0.1% of the negative control). The evaluation was carried out analogously to Example 1.

### Example 3

shows the phages after measurement using Hoechst stain. For the experiment, commercial microtiter plates (Greiner Bio-one; Sensoplate Plus) containing 384 reaction chambers (RCs) were used. First of all, the surface of the microtiter plate was constructed. For this purpose, the plate was placed into a desiccator in which a bowl containing 5% APTES in toluene was situated. The desiccator was flooded with argon and incubated for one hour. Thereafter, the bowl was removed and the plate was dried under vacuum for 2 hours. 20 μl of a 2 mM solution of SC-PEG-CM (MW 3400; Laysan Bio) in deionized H₂O were filled into the reaction chambers of the dry plate and incubated for 4 hours. After the incubation, the RC was washed three times with water and then incubated with in each case 20 μl of an aqueous 200 mM EDC solution (1-ethyl-3-(3-dimethylaminopropyl)carbodiimide; Sigma) and with 50 mM NHS (N-hydroxysuccinimide, Sigma) for 30 minutes. The plate was washed again with three times with deionized water. Thereafter, the RCs were coated with clone anti-gp8-E1 (prod. #ABIN793840, lot #77410, antibodies-online.com) antibodies as capture molecule (20 μl; μg/ml in PBS; 1 hour). Thereafter, the RC was treated with the wash program consisting of, in each case, washing and complete suction three times with TBS containing 0.1% Tween 20 and TBS. In the next step, the RCs were coated overnight with 50 μl of Smartblock (Candor Bioscience GmbH) at room temperature (RT) and, after this time had passed, were again subjected to washing and complete suction three times using tris(hydroxymethyl)aminomethane-buffered saline (TBS; pH=7.4). The samples were diluted sequentially in tris(hydroxymethyl)aminomethane (TRIS) buffer containing the dye Hoechst stain (1 μg ml-1) and incubated for one hour. Thereafter, 15 μl of sample were in each case loaded in RCs in triplicate and incubated at RT for 1 hour. After the incubation, the RCs were washed three times with TBS and, after this time had passed, the plate was washed 3 times with TBS. After complete suction, the RCs were filled with 20 μl of TBS and the plate was sealed a film.

The measurement was carried out in a TIRF microscope (Leica) with a 100× oil immersion objective. For this purpose, the glass base of the microtiter plate was generously coated with immersion oil and the plate was introduced into the automated stage of the microscope. Thereafter, what was consecutively recorded per RC at 5×5 positions was one image in the fluorescence channel (excitation/emission=405/450 nm). What was selected was the maximum laser output (100%), an exposure time of 500 ms and a gain value of 800. The image data were then evaluated. For the channel, an intensity threshold was set at 4000 grayscales. In the evaluation step, the intensity threshold was first applied for each image in each channel and images of the same position were then compared with one another in both values. What were counted per image were only those pixels in which, in both channels, the pixel at the exact same position is above the intensity threshold of the channel. Lastly, the number of pixels is averaged over all images in each RC and, afterwards, the mean values of the average pixel numbers of the replicate values are ascertained and the standard deviation is specified.

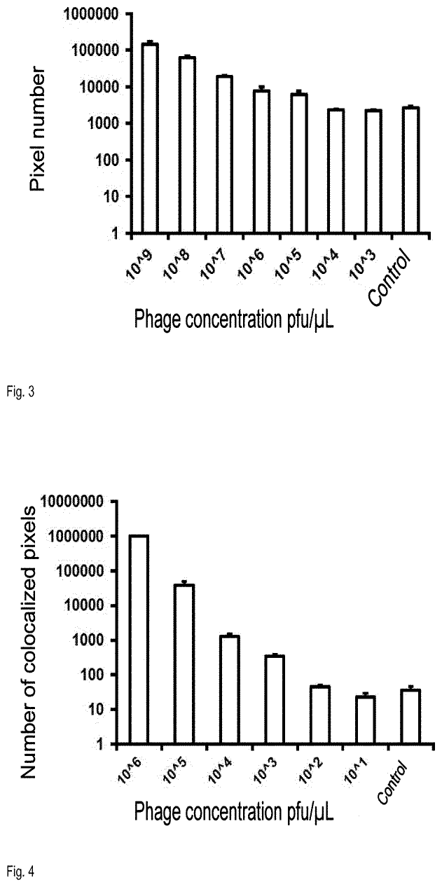

The results are summarized in FIG. 3.

FIG. 3 shows a concentration series of serially diluted M13 phages (Ph.D.-7 Phage Display Library; prod. #E8102L, lot #0061005, New England BioLabs) in TBS containing Hoechst stain. The figure shows a concentration-dependent relationship of M13 phages over log dilution steps (10\hat{ }9-10\hat{ }5), and the last two concentration steps could no longer be distinguished from the background.

What is clearly shown is the suitability of Hoechst stain as dye adhering to DNA and RNA in the virus assay for staining of the virus particles. It would thus be possible to detect intact viruses, or to distinguish between empty coats and coats containing DNA and/or RNA.

### Example 4

FIG. 4 shows the phages from FIG. 3 after the measurement using Höchst stain. The RCs of the microplate from Example 3 were washed three times with TBS and were incubated for 1 hour with the detection antibodies, 1.25 μg/ml RL-ph1 (prod. #LS-C146750, LifeSpan BioScience) labeled with CF488 fluorescent dye and 1.25 μg/ml LRL-ph2 (prod. #LS-C146751, lot #76955, LifeSpan BioScience) labeled with CF633 fluorescent dye. After washing three times with TBS, the samples were measured TIRF microscope (Leica) with a 100× oil immersion objective. For this purpose, the glass base of the microtiter plate was generously coated with immersion oil and the plate was introduced into the automated stage of the microscope. Thereafter, what was consecutively recorded per RC at 5×5 positions was in each case two images in two fluorescence channels (excitation/emission=635/705 nm and 488/525 nm). For both channels, what was selected was the maximum laser output (100%), an exposure time of 500 ms and a gain value of 800. The image data were then evaluated. For this purpose, intensity thresholds for each channel were ascertained on the basis of the negative control. For said threshold, all images of the negative control were averaged for each channel and what was ascertained was that intensity value above which only 0.1% of the total pixels (ergo 1000 pixels) are present. In the evaluation step, the intensity threshold was first applied for each image in each channel and images of the same position were then compared with one another in both values. What were counted per image were only those pixels in which, in both channels, the pixels at the exact same position were above the intensity threshold of the channel. Lastly, the number of pixels was averaged over all images in each RC and, afterwards, the mean values of the average pixel numbers of the replicate values were ascertained and the standard deviation was specified.

The experiment shows both the concentration-dependent relationship of M13 phages over 4 log dilution steps (10\hat{ }6-10\hat{ }3). It shows that a subsequent staining with fluorescently labeled antibodies is possible. Furthermore, the experiment shows that microtiter plates with PEG coating achieve approximately the same sensitivity as in the case of 3D NHS plates with a shorter incubation time (cf. FIG. 2).

### Example 5

The experiment was carried out in commercially available 3D NHS microtiter plates (PolyAn GmbH) containing 384 reaction chambers (RCs). The RCs of the microtiter plates were coated with anti-VSV-G antibody P5D4 (Sigma) as capture molecule (15 μl; 10 μg/ml in 100 mM MES, pH=4.7; overnight). Thereafter, the RC was treated with the wash program consisting of, in each case, washing and complete suction three times using phosphate-buffered saline (PBS) containing 0.1% Tween 20 and PBS. In the next step, the RCs were coated with 50 μl of Smartblock (Candor Bioscience GmbH) at room temperature (RT) for 1 h and, after this time had passed, were again subjected to washing and complete suction three times using tris(hydroxymethyl)aminomethane-buffered saline (TBS; pH=7.4). The samples were sequentially in tris(hydroxymethyl)aminomethane-buffered saline (TBS) incubated for one hour. Thereafter, 15 μl of sample were in each case loaded in RCs in triplicate and incubated at RT for 1 hour. After the incubation, the RCs were subjected to washing and complete suction three times using TBS and were admixed with 15 μl of detection antibodies. The detection antibodies were in each case labeled with one type of fluorescent dye. Anti-VSV-G antibodies P5D4 (Sigma) were in each case labeled with CF488 and with CF633. The detection antibodies were diluted together in TBS to give a final concentration of 1.25 ng/ml for each antibody. 15 μl of antibody solution were loaded per RC and incubated at RT for 1 h. After this time had passed, the plate was washed 3 times with TBS. After complete suction, the RCs were filled with 20 μl of TBS and the plate was sealed a film.

The measurement was carried out in a TIRF microscope (Leica) with a 100× oil immersion objective. For this purpose, the glass base of the microtiter plate was generously coated with immersion oil and the plate was introduced into the automated stage of the microscope. Thereafter, what was consecutively recorded per RC at 5×5 positions was in each case two images in two fluorescence channels (excitation/emission=635/705 nm and 488/525 nm). For both channels, what was selected was the maximum laser output (100%), an exposure time of 500 ms and a gain value of 800. The image data were then evaluated. For this purpose, intensity thresholds for each channel were ascertained on the basis of the negative control. For said threshold, all images of the negative control were averaged for each channel and what was ascertained was that intensity value above which only 0.1% of the total pixels (ergo 1000 pixels) are present. In the evaluation step, the intensity threshold was first applied for each image in each channel and images of the same position were then compared with one another in both values. What were counted per image were only those pixels in which, in both channels, the pixels at the exact same position were above the intensity threshold of the channel. Lastly, the number of pixels was averaged over all images in each RC and, afterwards, the mean values of the average pixel numbers of the replicate values were ascertained and the standard deviation was specified.

The results are summarized in FIG. 5.

FIG. 5 shows a concentration series of serially diluted virus particles of an HIV safety strain (for the biosynthesis, see J Mol Biol. 2017 April 21; 429(8): 1171-1191. doi: 10.1016/j.jmb.2017.03.015., which contains a VSV glycoprotein in the lipid membrane) diluted in TBS from cell culture medium. The figure shows a linear relationship between the measurement signal and the concentration of virus particles over 3 log dilution steps and a distinguishability of the lowest concentration in relation to the background. Since a virus particle with lipid coat is concerned here, the method according to the invention can be used on for the analysis of this taxonomic group.

### Example 6

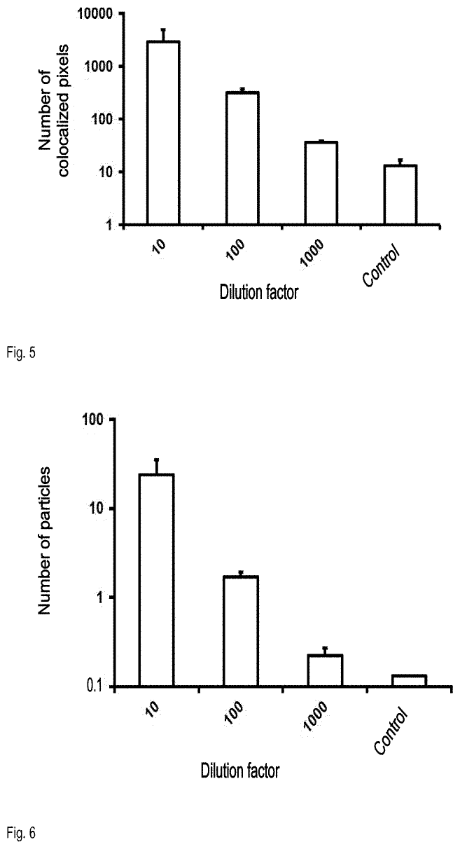

FIG. 6 shows the number of virus particles per μm² in the individual dilution steps of the data from FIG. 5. This alternative evaluation of the data allows absolute counting of virus particles and is thus independent of a standard. For the evaluation, what were multiplied for each recording were the grayscale values of each corresponding pixel from both images in which fluorescence color channels were obtained. The resultant image, or the grayscale value matrix, was smoothed on the basis of a 2D Gaussian function (“imgaussfilt”) with a standard deviation of 4 pixels. This was followed by the determination of the local maxima on the basis of the function “imregionalmax”. The number of local maxima above a threshold corresponds here to the number of particles, the threshold corresponding to the threshold ascertained in FIG. 5. In this way, the number of particles was determined for each recording and, finally, the mean value was formed over RCs of the same concentration.

1.-15. (canceled)

16. A method for quantitatively and/or qualitatively determining virus particles containing at least one binding site for capture molecules and at least one binding site for probes, wherein the method comprises:
(a) immobilizing capture molecules on a substrate,
(b) contacting the virus particles with the capture molecules,
(c) immobilizing the virus particles on a substrate by binding to capture molecules,
(d) contacting the virus particles with the probes and
(e) binding the probes to the virus particles,
and wherein the probes are capable of emitting a specific signal and (b) and (d) can be carried out simultaneously or (d) can be carried out before (b).

17. The method of claim 16, wherein a spatially resolved determination of a probe signal is carried out.

18. The method of claim 16, wherein the virus particles are selected from virus, virion, bacteriophage, parts or fragments of the former.

19. The method of claim 16, wherein the substrate is composed of a material selected from plastic, silicon, silicon dioxide.

20. The method of claim 16, wherein the substrate is composed of glass.

21. The method of claim 16, wherein the substrate has a hydrophilic surface prior to (a).

22. The method of claim 21, wherein a hydrophilic layer is applied to the substrate prior to (a).

23. The method of claim 22, wherein the hydrophilic layer is selected from PEG, poly-lysine, dextran, derivatives thereof.

24. The method of claim 22, wherein prior to application of the hydrophilic layer the substrate is hydroxylated and functionalized with reactive groups (amino groups).

25. The method of claim 24, wherein functionalization with amino groups is achieved by contacting the substrate with APTES (3-aminopropyltriethoxysilane).

26. The method of claim 25, wherein the substrate is contacted with APTES in the gas phase.

27. The method of claim 24, wherein functionalization with amino groups is achieved by contacting the substrate with ethanolamine.

28. The method of claim 16, wherein the capture molecules are covalently bonded to the substrate or a coating thereof.

29. The method of claim 16, wherein the binding sites of the virus particles are epitopes and the capture molecules and probes are antibodies or aptamers or combinations thereof.

30. The method of claim 16, wherein the probes are labeled with fluorescent dyes.

31. The method of claim 16, wherein detection is carried out by spatial-resolution fluorescence microscopy.

32. A kit for carrying out the method of claim 16, wherein the kit comprises one or more of a substrate, optionally with hydrophilic surface, capture molecules, probes, substrate with capture molecules, solutions, buffers.
